(19) United States
(12) Patent Application Publication
(10) Pub. No.: US 2004/0219232 A1
(43) Pub. Date: Nov. 4, 2004

(54) Methods and compounds for treating malabsorption diseases and inflammatory conditions of the gastrointestinal tract

(75) Inventor: James M. Lipton, Woodland Hills, CA (US)

Correspondence Address: PERKINS COIE LLP, POST OFFICE BOX 1208, SEATTLE, WA, 98111-1208, US

(73) Assignee: Zengen, Inc.

(21) Appl. No.: 10/774,271

(22) Filed: Feb. 6, 2004

Related U.S. Application Data
(60) Provisional application No. 60/445,542, filed on Feb. 6, 2003.

(51) Int. Cl. A61K 35/78

(52) U.S. Cl. 424/725

(57) ABSTRACT

A method of treatment for malabsorption diseases is disclosed. Use of α-MSH and/or its derivatives in an amount equal to 25 mg/kg of body weight in combination with artichoke leaf extract in an amount equal to 300 to 1000 mgs, for those afflicted with a malabsorption disease is discussed and it is shown that the local action of α-MSH reduces the inflammatory component as well as the deleterious effects of celiac disease in intestinal mucosa exposed to gluten or gliadin.

### PRIORITY CLAIM

This application claims priority to U.S. Provisional Application Ser. No. 60/445,542, filed on Feb. 6, 2003, and is incorporated by reference as if fully set forth herein, including drawings.

### FIELD OF THE INVENTION

The present invention relates to the field of prevention and treatment of malabsorption and inflammatory conditions of the intestines.

### BACKGROUND OF THE INVENTION

The gastrointestinal tract (“GIT”) digests and absorb nutrients including macromolecules required for energy or micronutrients such as trace elements and vitamins. The absorptive function of the GIT ranges from the μg level to kg level. For example 1 μg of vitamin B₁₂ may be absorbed daily with respect to a highly specific and complex process in the terminal ileum. Conversely, the GIT may digest, solubilize,

Beyond the net absorption of dietary components, the GIT has a large daily internal circulation of water, electrolytes and bile salts that are secreted in gastric juice, pancreatic juice, bile and succus entericus and then reabsorbed in the small intestine (duodenum-jejunum-ileum) and colon (cecum-ascending-transverse-descending-sigmoid-rectum). Andreoli, Carpenter, Plum Smith, Cecil Essentials of Medicine pp.267-270, (W. B. Saunders Co. 1986). The GIT must first prepare for the later absorption of the basic mixtures of nutrients and non-nutrients ingested as bolus, or food. The preparatory processes of digestion consist, basically, of the controlled release into the intestine of food that has been fragmented to small particle size by the grinding action of the stomach; the release of the appropriate amount of enzymes, cofactors, bicarbonate, detergents and water into the lumen; and further digestion of food to disaccharides and oligopeptides by brush border bound enzymes. Absorption follows at various levels of the duodenum, jejunum and ileum. Id.

The surface area available to the GIT is enormous. The overall length of the small intestine is minimal compared to the surface area which, in a normal adult human, is about the area of a tennis court. This added surface area is supplied by the intestinal villi and microvilli.

Absorption is a highly complex and coordinated process and in view of the many complexities is vulnerable to a large number of disorders that result in maldigestion or malabsorption. These many disorders may act specifically at the site of insult or more globally. The primary focus of the present invention is the malabsorptive diseases.

Many disorders of the GIT can be listed in the category of malabsorption. This list includes, but is not limited to, celiac sprue (gluten-sensitive enteropathy), Crohn's disease, abetalipoproteinemia, Hartnup's disease, tropical sprue, bacterial overgrowth syndrome, cystinuria, monosaccharide absorptive defects, Whipple's disease and infective, immunologic or allergic injury. These disorders vary from specific genetic defects to the acquired diffuse mucosal diseases and represent a broad spectrum of conditions with varying etiologies. Clinical manifestations are equally diverse. Id.

Treatments of the various malabsorption diseases, for the most part, match the disease entity. For example, bacterial overgrowth is battled with antibiotics, as is Whipple's disease. Celiac sprue, on the other hand, is treated with a gluten free diet. Most treatments are symptomatic as opposed to prophylactic. Patients with chronic malabsorption diseases would benefit from a treatment that both addressed prevention and maintenance.

This is especially true in celiac disease, or gluten-sensitive enteropathy. This disease is a familiar, chronic familial disorder associated with a life-long sensitivity to dietary gluten, or more specifically, gliadin (a component of gluten). The disease is especially significant in that gluten is a predominant protein found in wheat and wheat products, barley, rice and, in smaller amounts, oats. When gluten is ingested by a person with gluten-sensitivity, a diffuse mucosal injury results that is characteristic of the disease, but in no way specific. Histologically, the villi are shortened and blunted in one with celiac disease. The blunted and shortened villi result in a less absorptive surface area. The crypts, the areas between the villi, are hyperplastic and the lamina propria, the layer of connective tissue underlying the epithelium of a mucous membrane, is usually heavily infiltrated with lymphocytes. Ferguson A, Murray D., Quantitation of intraepithelial lymphocytes in human jejunum, Gut 1971; 12:988-994.

Pathogenically, the etiology has yet to be fully established. Evidence suggests an immunological mechanism. Andreoli, Carpenter, Plum Smith, Cecil Essentials of Medicine pp. 267-270, (W. B. Saunders Co. 1986). Environmental and genetic causes are also important. Braunwald, Fauci, Kasper, Longo, Jameson Harrison's Principles of Internal Medicine, pp. 1165-1674, (15^{th} Ed. McGraw Hill, 2001). Patients with a celiac sprue have a high prevalence for the histocompatibility types HLA-B8 and Dw3. This suggests a linkage on chromosome 6 and strengthens the possibility that the disease may be of immunological origin. Regardless of the origin, however, it is known that the lesions connected with celiac sprue are locally produced. Id.

The local lesions of celiac sprue occur mostly in the duodenal-proximal jejunal mucosa. The most important complication of celiac disease is the development of a malignancy. There is an increased incidence of both gastrointestinal and nongastrointestinal neoplasms as well as lymphoma in patients with celiac disease. Id. Ulceration is another complication of celiac disease. Because celiac disease is a multifactorial disease and exhibits, among other complications, both nutritional and inflammatory complications, a treatment that responds to the multifactorial nature of the disease is needed. The same is true for the malabsorption diseases in general.

The specific categorization of celiac disease is a disorder characterized by intolerance to gluten, malabsorption, and abnormal small bowel structure. Schuppan D, Current concepts of celiac disease pathogenesis, Gastroenterology 2000; 119:234-242. The disease is believed to be caused by a T cell-mediated hypersensitivity reaction to certain A-gliadin polypeptides in genetically susceptible patients. The small intestinal mucosa of affected patients shows crypt cell hyperplasia and villous flattening, which are the pathognomonic lesions of the disease. Paulley L W, Observations on the aetiology of idiopathic steatorrhoea, Br Med J 1954; 2:1318-1321; Yardley J H, Bayless T M, Norton J H, Hendrix T R., Celiac disease: a study of the jejunal epithelium before and after a glutenfree diet, New Engl J Med 1962; 267:1173-1179. The lamina propria is infiltrated with lymphocytes, macrophages, and plasma cells. Inflammatory mediators released by infiltrating cells likely contribute to development of the disease. Ferguson A, MacDonald T T, McClure J P, Holden R J, Cell-mediated immunity to gliadin within the small-intestinal mucosa in coeliac disease, Lancet 1975; 1:895-897. Immunohistochemical analysis of celiac mucosa showed increased number of cells positive for interferon γ (IFN-γ) in the lamina propria (Nilsen E M, Jahnsen F L, Lundin K E, Johansen F E, Fausa O, Sollid L M, Jahnsen J, Scott H, Brandtzaeg P., Gluten induces an intestinal cytokine response strongly dominated by interferon gamma in patients with celiac disease, Gastroenterology 1998; 115:551-563) and for interleukin 6 (IL-6) and tumor necrosis factor α (TNF-α) both in the lamina propria and epithelium. Przemioslo R T, Kontakou M, Nobili V, Ciclitira P J, Raised pro-inflammatory cytokines interleukin 6 and tumor necrosis factor a in coeliac disease mucosa detected by immunohistochemistry, Gut 1994; 35:1398-1403. Further, gliadin stimulation of duodenal specimens in vitro elicited expression of the pro-inflammatory cytokines, including IFN-γ, IL-6 and TNF-α. Nilsen E M, Jahnsen F L, Lundin K E, Johansen F E, Fausa O, Sollid L M, Jahnsen J, Scott H, Brandtzaeg P., Gluten induces an intestinal cytokine response strongly dominated by interferon gamma in patients with celiac disease, Gastroenterology 1998; 115:551-563; Beckett C G, Dell'Olio D, Shidrawi R G, Rosen-Bronson S, Ciclitira P J., Gluten-induced nitric oxide and pro-inflammatory cytokine release by cultured celiac small intestinal biopsies, Eur J Gastroenterol Hepatol 1999; 11:529-535.

Currently, treatment is directed to removing the source of gliadin from the diet. Thus, once lesions have been created in the intestinal tract, they remain inflamed and painful until there is healing via the body's reparative system. Unfortunately, lesions may progress before the body has healed them. These lesions may progress on to chronic ulceration or to malignancy. The most common medications used to treat inflammation Non-Steroidal Anti-Inflammatory Drugs (NSAIDs) are most often contraindicated in patients with any type of history of gastritis, or history of ulcer. Steroids, a more aggressive anti-inflammatory treatment, pose the additional risks, including that an ulcer may become infected but go unnoticed due to the well-known side effect of systemic steroids in masking infections. A treatment is needed that is natural and does not interfere with the body's natural defense mechanisms. Such a treatment compound and method is disclosed in this invention.

### SUMMARY OF THE INVENTION

The current invention is directed to treatment of malabsorption diseases and inflammatory disorders of the GIT using a combination of artichoke leaf extract and the peptide, α-melanocyte stimulating hormone. Alpha-melanocyte stimulating hormone (“α-MSH”) has potent anti-inflammatory activity and artichoke leaf extract is known for its anti-cholesterol anti-gastritis effects. Recent research by the inventors has shown that α-MSH and its receptors participate in the anti-inflammatory response in the duodenal mucosa of celiac patients. The same is contemplated for derivatives of α-MSH. α-MSH and/or its derivatives may be used in medicinal preparations to treat and help prevent the complications associated with celiac disease and other inflammatory conditions of the GIT.

The presence of α-MSH and its receptors in celiac mucosa suggests the presence of a local reaction to control the inflammatory response elicited by gliadin. The inhibitory effects of synthetic α-MSH on mucosal IL-6 production is consistent with such a local modulatory reaction based on the peptide. This local action may be beneficially controlled or modified with medicinal administration of α-MSH and/or its derivatives. In this respect, the invention may be used as a preventive measure as well as a treatment during an attack. Further, it has been shown that artichoke leaf extract reduces symptoms of irritable bowel syndrome.

It is contemplated that artichoke leaf extract and α-MSH and/or its derivatives will be administered via oral, anal, mucosal or paranteral routes. Accordingly, artichoke leaf extract and α-MSH and/or its derivatives will be administered using tablets, capsules, powders, suppositories, aerosolized sprays and injectables.

### DESCRIPTION OF FIGURES

FIG. 1. A-MSH release by duodenal mucosa specimens incubated for 24 h in vitro. Open bars denote incubation in medium alone; black bars represent incubation in medium+gliadin. Spontaneous α-MSH production was significantly greater in untreated celiac patients relative to both control subjects and treated celiac patients. Co-incubation of mucosa specimens with gliadin elicited further release of α-MSH only in untreated celiac patients. (In this and following figures bars represent mean f SE).

*p<0.05: spontaneous release in celiac patients vs. controls and treated celiac patients, and gliadin stimulated vs. spontaneous release in celiac patients.

FIG. 2. Immunohistochemical detection of α-MSH in a duodenal specimen from a patient with untreated celiac disease. (Original magnification X220).

FIG. 3. Agarose gel electrophoresis of representative RT-PCR amplified mRNA for POMC and MCIR in duodenal specimens from: a normal subject (1^{st} lane), an untreated celiac patient (2nd lane), and a treated celiac patient (3rd lane). X174 RF DNAIHae III fragments were used as molecular weight markers (4th lane). A) POMC mRNA 260 by amplified fragment. B) MC1R mRNA 416 by amplified fragment. C) G3PDH mRNA 980 by amplified fragment. The indicated size (base pairs) of the amplification products matched that predicted from the position of the primer pairs.

FIG. 4. Immunohistochemical detection of MCIR in normal duodenal mucosa and in duodenal specimens from two patients with celiac disease. Arrows denote immunoreactive cells. A) Normal villi with no MCIR immunoreactive cells on the villous surface. B) Specimen from the same subject showing goblet cells of glands weakly stained with the anti-MCIR antibody. C) MCIR immunoreactive cells on the surface of flattened villi in a patient with untreated celiac disease. D) Specimen from the same subject showing intense MCIR immunoreactivity of goblet cells. E) and F) Duodenal mucosa specimens from a celiac patient treated with an embodiment of the invention. Relative to the untreated patient, immunoreactive cells are fewer and intensity of staining is lower. However, distribution of positive cells is similar to that of the untreated patients, involving the villous surface. (Original magnification ×110).

FIG. 5. Immunohistochemical detection of MC5R in duodenal specimens from: A) normal subject, B) patient with untreated celiac disease, and C) treated celiac patient. Scattered cells were marked with the antiserum against MC5R; there were no substantial differences among normal, celiac, or celiac subjects treated with an embodiment of the invention. (Original magnification ×220).

FIG. 6. Effect of gliadin on IL-6 production by duodenal specimens incubated for 24 h in vitro (open bars), and influence of α-MSH on such production (black bars). IL-6 production was markedly enhanced in untreated celiac patients, although a small increase occurred also in the other two groups. Coincubation of gliadin-stimulated duodenal specimens with α-MSH (10-5 M) significantly decreased IL-6 production.

(* p<0.05; ** p<0.01)

### DETAILED DESCRIPTION OF THE INVENTION

All references cited are fully incorporated as if fully set forth herein. Research by the inventors, discussed more fully below, has shown that human duodenal mucosa is a source of α-MSH and that the duodenal mucosa expresses melanocortin receptors. Both production of α-MSH and MC1R immunoreactivity, were considerably enhanced in duodenal samples from untreated celiac patients. That observation suggested that locally produced α-MSH, a potent anti-inflammatory agent, could modulate inflammation and possibly reduce epithelial damage. When gliadin-stimulated celiac mucosa was incubated with synthetic α-MSH there was a substantial reduction in IL-6 production. Thus, it is contemplated that the invention may be used as a therapeutic measure before, during and after the complications of a malabsorption condition have arisen.

Although understanding of the mechanism of anti-inflammatory action of α-MSH is incomplete, part of its anti-inflammatory influence is exerted through reduced production of inflammatory mediators by host cells. α-MSH inhibits the inflammatory cascade at many sites: it reduces production of NO (Star R A, Rajora N, Huang J, Stock R C, Catania A, Lipton J M., Evidence of autocrine modulation of macrophage nitric oxide synthase by alpha-melanocyte-stimulating hormone, Proc Natl Acad Sci USA 1995; 92:8016-8020), proinflammatory cytokines (Lipton J M, Catania A., Anti-inflammatory actions of the neuroimmunomodulator α-MSH, Immunol Today 1997; 18:140-145), monocyte chemoattractant protein 1 (MCP-1), and interleukin 8 (IL-8), and markedly decreases neutrophil chemotaxis in vivo and in vitro. Mason MJ, Van Epps D., Modulation of IL-1, tumor necrosis factor, and C5a-mediated murine neutrophil migration by a-melanocyte-stimulating hormone. J Immunol 1989; 142:1646-1651; Catania A, Rajora N, Capsom F, Minonzio F, Star R A, Lipton J M, The neuropeptide α-MSH has specific receptors on neutrophils and reduces chemotaxis in vitro, Peptides 1996; 17:675-679. These effects of the peptide are exerted, at least in part, through inhibition of activation of the nuclear factor NF-κB, a pivotal transcription factor for genes that encode proinflammatory cytokines, chemokines, and adhesion molecules. Manna S K, Aggarwal B B, α-Melanocyte-stimulating hormone inhibits the nuclear transcription factor NT-KB activation induced by various inflammatory agents, J Immunol 1998; 161:2873-2880; Ichiyama T, Zhao H, Catania A, Furukawa S, Lipton J M, α-Melanocyte-stimulating hormone inhibits NF-KB activation and IKBa degradation in human glioma cells and in experimental brain inflammation, Exp Neurol 1999; 157:359-365.

It is known that the influences of melanocortin peptides, including A-MSH, are mediated through activation of melanocortin receptors (MCRs). The cloning and characterization of the MCR genes (MC1R through MC5R) have led to a great advancement in understanding the biological effects of melanocortins. Abdel-Malek Z A, Melanocortin receptors: their functions and regulation by physiological agonists and antagonists, Cell Mol Life Sci 2001; 58:434-441. Each receptor is the product of a separate gene and collectively they represent a distinct family of seven transmembrane domain G-protein coupled receptors. Activation of the MCRs by binding of their specific agonists results in stimulation of cAMP formation. Although the function of each MCR is not completely known, it is clear that these receptors have different affinities for the individual melanocortin peptides and mediate separate effects. Virtually all cells involved in inflammatory responses express one or more subtypes of melanocortin receptors.

Human monocyte/macrophages express MC1R, the receptor with the highest affinity for α-MSH (Taherzadeh S, Sharma S, Chhajlani V, Gantz I, Rajora N, Demitri M T, Kelly L, Zhao H, Catania A, Lipton J M, α-MSH and its receptors in regulation of inflammatory tumor necrosis factor-a (TNF-α) by human monocyte/macrophages, Am J Physiol 1999; 276:R1289-R1294), which is also expressed by human neutrophils (Catania A, Rajora N, Capsom F, Minonzio F, Star R A, Lipton J M, The neuropeptide α-MSH has specific receptors on neutrophils and reduces chemotaxis in vitro, Peptides 1996; 17:675-679), and endothelial cells. Abdel-Malek Z A, Melanocortin receptors: their functions and regulation by physiological agonists and antagonists, Cell Mol Life Sci 2001; 58:434-441. MC3R and MC5R expression has also been found in human monocytes.

The current invention is based on findings by the inventors of the occurrence of an anti-inflammatory circuit based on α-MSH and its receptors in celiac mucosa. Locally produced and/or circulating α-MSH can exert anti-inflammatory influences through binding to MC1R receptors. Several data support this idea: a) celiac mucosa specimens incubated in vitro produce greater concentrations of α-MSH, both spontaneously and after gliadin stimulation; b) MC1R, the receptors with the greatest affinity for A-MSH are hyperexpressed in untreated celiac patients; c) IL-6 production is inhibited by addition of α-MSH to culture medium.

Anti-inflammatory circuits based on α-MSH and its receptor(s) are probably widespread in tissues. Functionally equivalent derivatives of α-MSH may react within these same circuits. Previous research indicated the existence of an autocrine loop in human macrophages: such cells produce α-MSH and express the MC1R; immunoneutralization of MC1R enhances production of TNF. Local production of α-MSH likewise occurs in synovium of rheumatoid arthritis patients. Catania A, Gerloni V, Procaccia S, Airaghi L, Manfredi M G, Lomater C, Grossi L, Lipton J M, The neuropeptide α-MSH in synovial fluid of patients with rheumatic diseases: comparisons with other anticytokine molecules, Neuroimmunomodulation 1994; 1:321-328.

Inflammatory reactions in body tissues are modulated by endogenous molecules that reduce production of inflammatory mediators and limit tissue damage. Therefore, the extent of tissue injury depends upon the amount of inflammatory products released and the effectiveness of general and local anti-inflammatory reactions. The data relied on here suggest that α-MSH and its receptors contribute to the anti-inflammatory reaction in celiac mucosa. In selected cases of refractory celiac disease or when compliance to the gluten free diet is poor, treatment with exogenous peptides might be considered. In this regard, previous research showing that α-MSH is highly effective in preclinical treatment of inflammatory bowel disease (Rajora N; Boccoli G, Catania A, Lipton J M., α-MSH modulates experimental inflammatory bowel disease, Peptides 1997; 18:381-385; Oktar B K, Ercan F, Yegen B C, Alican I, The effect of a-melanocyte stimulating hormone on colonic inflammation in the rat, Peptides 2000; 21:1271-1277) is particularly encouraging.

Although the inflammatory component in celiac disease is well characterized, there is less information about endogenous molecules that reduce such inflammation. α-MSH is an established component in the host anti-inflammatory reactions. Catania A, Lipton J M, α-Melanocyte stimulating hormone in the modulation of host reactions. Endocr Rev 1993; 14:564-576; Catania A, Airaghi L, Colombo G, Lipton J M, α-MSH in normal human physiology and disease states, Trends Endocrinol Metab 2000; 11:304-308. This molecule, produced by post-translational processing of pro-opiomelanocortin (POMC), is a 13 amino acid peptide highly conserved across phylogeny and widely expressed in tissues. Eberle A N, The Melanotropins, Basel (ed. S. Karger) 1988. The peptide is produced by the pituitary and by many extrapituitary cells, including monocytes, astrocytes, gastrointestinal cells, and keratinocytes. Endogenous α-MSH modulates fever and inflammation. Given in pharmacological concentrations, α-MSH is extremely effective in preclinical treatment of local and systemic inflammatory disorders including inflammatory bowel disease, sepsis syndrome, and rheumatoid arthritis. Lipton J M, Ceriani G, Macaluso A, McCoy D, Carries K, Biltz J, Catania A, Anti-inflammatory effects of the neuropeptide alpha-MSH in acute, chronic, and systemic inflammation, Ann N Y Acad Sci 1994; 741:137-148; Lipton J M, Catania A, Anti-inflammatory actions of the neuroimmunomodulator α-MSH, Immunol Today 1997; 18:140-145.

Although plasma concentrations of α-MSH increase in certain infectious or inflammatory disorders, (Catania A, Airaghi L, Colombo G, Lipton J M, α-MSH in normal human physiology and disease states, Trends Endocrinol Metab 2000; 11:304-308; Lipton J M, Catania A, Anti-inflammatory actions of the neuroimmunomodulator α-MSH, Immunol Today 1997; 18:140-145) and there is evidence of descending modulatory reactions stemming from MSH peptide receptors within the brain (Macaluso A, McCoy D, Ceriani G, Watanabe T, Biltz J, Catania A, Lipton J M, Anti-inflammatory influences of α-MSH molecules: central neurogenic and peripheral actions, J Neurosci 1994; 14:2377-2382), it is clear that the peptide produced at the site of an inflammatory process can exert its effects locally, in the absence of any significant change in circulating peptide. Synovial production of A-MSH in rheumatoid arthritis patients provides a good example of such local influences. Catania A, Gerloni V, Procaccia S, Airaghi L, Manfredi M G, Lomater C, Grossi L, Lipton J M, The neuropeptide α-MSH in synovial fluid of patients with rheumatic diseases: comparisons with other anticytokine molecules. Neuroimmunomodulation 1994; 1:321-328.

It is contemplated that derivatives of α-MSH will have analogous function. It is understood that derivatives of α-MSH means functionally equivalent peptides. For example, certain segments of α-MSH are known to have more potent anti-inflammatory and/or antipyretic properties. Thus, α-MSH derivative would include, among others, the partial segments of the 13 amino acid peptide. Specifically, α-MSH is composed of the following sequence of amino acids: SYSMEHFRWGKPV (SEQ ID. NO. 1). Thus, KPV (SEQ ID. NO. 2), which has been shown to be as potent or more potent than the parent α-MSH, is a functional equivalent. Further, similar efficacies, when compared to α-MSH, have been shown for MEHFRWGKPV (SEQ ID. NO. 3), HFRWFKPV (SEQ ID. NO. 4) and, as mentioned, KPV (SEQ ID. NO. 2). These derivatives of the parent α-MSH are functional equivalents. Inventors have also discovered that the sequence His-DNle-Arg-Trp-Phe-Lys-Pro-Val (SEQ. ID NO. 5) has increased potency over the parent α-MSH and its derivatives.

Further, it is understood that α-MSH and/or its derivatives may be used in this invention as a dimer. For example, the dimer of KPV is VPK-Ac-CC-Ac-KPV (SEQ ID. NO. 6). Moreover, it is known that substitution of certain amino acids with their stereo isomer results in a molecule with similar or greater efficacy. Thus, KDPV, where DP is the D-isomer of proline, is an effective functional equivalent of α-MSH. Substitutions with other equivalent amino acids may also be employed. For example, by substitutions at amino acid 4 and 7 in the α-MSH parent molecule, the resulting molecule becoming [Nle⁴,D-Phe⁷]-α-MSH (“NDP-α-MSH”), has been shown be a stable and potent functional equivalent of α-MSH. This is further described in Example 1, below.

Efficacy and duration of action of peptides may be additionally improved through protection of the functional groups of the peptides. This is a process well known and understood in the art. It is contemplated that the peptides of this invention will be protected by esterification or amidation of the —COOH to —COO-alkyl or —CONH₂. The preferred alkyl groups for the carboxy terminus include methyl and benzyl residues, yet other alkyl groups, such as ethyl, propyl, butyl, p-nitrobenzyl or p-methoxybenzyl groups can be used.

Likewise, the amino terminus is protected by acylation via introduction of a carboxyl group such as an acetyl group, t-butyloxycarbonyl group, t-amyloxycarbonyl group, p-nitrobenzyloxycarbonyl group, toysl or formyl group. Such amidation, esterification, and acylation are disclosed in U.S. Pat. No. 5,028,592 to Lipton.

In one embodiment of the invention, α-MSH, and/or its derivatives, may be administered parenterally. For this embodiment, pharmaceutical preparations of the tripeptide, or its derivatives, may be generally obtained by combining the active ingredient in combination with pharmaceutically acceptable buffers, dilutents, stabilizers, and the like. In a preferred composition, approximately 100 to 500 mg of the active ingredient is dispersed into about 1-7 ml of sterile, isotonic saline, including a pharmacologically accepted buffer to maintain pH at about neutral. Parenteral administration may be desired when a GIT disease manifests pronounced stetorrhea, diarrhea or nausea/vomiting.

Due to its small size, membrane permeability and relatively acid-stable structure, it will be recognized that the α-MSH, and/or its derivatives, may be administered orally, through the oropharanx or nasopharanx via an appropriate inhalant apparatus or anally with the use of a suppository. For GIT disease or disorders, an oral route of administration is preferred.

Pharmacologically effective concentrations of these peptides may be incorporated into commercial formulations of parenterals, tablets, capsules, powders for mixing with water, or atomized sprays. See, in general Remington 's Pharmaceutical Sciences (Mack Publishing Co., 18^{th} ed. 1990).

In one aspect of this invention a combination of artichoke leaf extract with α-MSH and/or its derivatives is contemplated. Artichokes, or Cynara scolymus, contain the active ingredient cynarin. Walker A F, Middleton R W, Petrowicz O. Cynarin is the active ingredient in artichoke leaf extract. Artichoke leaf extract and the products thereof are found in most natural food and vitamin stores. The Hugh Sinclair Unit of Human Nutrition, School of Food BioSciences, University of Reading, UK. <food@afnovell.reading.ac.uk> It has been shown that cynarin is a natural therapeutic helpful in the treatment of abdominal problems, irritable bowel syndrome, for example.

Irritable bowel syndrome (IBS) is a problem reported to affect 22% of the general population. IBS is characterized by abdominal pain and altered bowel habit but has so far defied elucidation of its pathogenesis and proved difficult to treat. There is a growing body of evidence that indicates therapeutic properties for artichoke leaf extract (ALE). Dyspepsia and elevated cholesterol are the conditions for which the herb is specifically indicated. Many symptoms overlap between dyspeptic syndrome and IBS and this has given rise to the notion that ALE may have potential for treating IBS.

In 1996 a German study using 553 patients with digestive disorders was performed using ALE. Patients were given one or two 320 mg capsules of ALE three times a day for six weeks. As many as 80% of those studied reported overall relief. Kennedy, J. Natural Health, 2002. <http://www.atlantisherbs.com/Articles/artichokeIBS.htm>. Studies using ALE have been performed that are specific to IBS and have shown an average improvement of symptoms of 70%. Analysis of the data from the IBS study revealed significant reductions in the severity of symptoms and favorable evaluations of overall effectiveness by both physicians and patients. Furthermore most patients studied rated ALE as better than or at least equal to previous therapies administered for their symptoms, and the tolerability of ALE was very good. These results provide support for the notion that ALE has potential value in relieving IBS symptoms and may be further enhanced used in combination of α-MSH and or its derivatives. The combination of ALE and His-DNle-Arg-Trp-Phe-Lys-Pro-Val (SEQ. ID NO. 5) may be the most potent.

Set forth below are examples of various formulations of the invention. As used below, the term “Active Ingredient” refers to A-MSH and/or its derivatives. In this application, the term derivatives means those derivative discussed in Example 1 below as well as any fragments of α-MSH or dimers of A-MSH or dimers of the fragments, thereof. Dosages given in mg for the active ingredient are contemplated to be dosed at an average of 1 mg/kg of body weight for the individual to be treated. Thus, a 75 kg person would be given 75 mgs of the active ingredient. Preferably, the active ingredient is KPV (SEQ ID. NO. 1) or VPK-Ac-CC-Ac-KPV (SEQ ID. NO. 5).

An exemplary parenteral preparation comprises:

| | |
|---|---|
| Sterile Isotonic Saline | 1-7 cc |
| Pharmaceutically | In an amount adequate to |
| Accepted Buffer | maintain pH of about neutral |
| Artichoke Leaf Extract | 320-1000 mg |
| Active Ingredient | 10-100 mg |

An exemplary formulation of a hard gelatinous tablet comprises:

| | | |
|---|---|---|
| Gelatine Bloom 30 | 70.0 | mg |
| Maltodextrin MD 05 | 108.0 | mg |
| di-α-tocopherol | 2.0 | mg |
| Sodium ascorbate | 10.0 | mg |
| Microcrystalline cellulose | 48.0 | mg |
| Artichoke Leaf Extract | 320-1000 | mg |
| Magnesium stearate | 2.0 | mg |
| Active Ingredient | 25.0 | mg |

An exemplary formulation of a hard tablet with α-MSH and/or its derivatives in combination with ALE comprises:

| | | |
|---|---|---|
| Anhydrous lactose | 130.5 | mg |
| Microcrystalline cellulose | 80.0 | mg |
| di-α-tocopherol | 2.0 | mg |
| Sodium ascorbate | 10.0 | mg |
| Polyvinylpyrrolidone K30 | 5.0 | mg |
| Artichoke Leaf Extract | 320-1000 | mg |
| Magnesium stearate | 2.0 | mg |
| Active Ingredient | 25.0 | mg |

### EXAMPLE I

### KPV Derivatives

This example illustrates the biological functional equivalents for, α-MSH; termed herein as its derivatives. Although the specific amino acid sequence described here is effective, it is clear to those familiar with the art that amino acids can be substituted or deleted without altering the effectiveness of the peptides. Further, it is known that stabilization of the α-MSH and/or its derivatives' sequence can greatly increase the activity of the peptide and that substitution of D-amino acid forms for L-forms can improve or decrease the effectiveness of the peptides. For example, a stable analog of α-MSH, [Nle⁴,D-Phe⁷]-α-MSH, which is known to have marked biological activity on melanocytes and melanoma cells, is approximately ten times more potent than the parent peptide in reducing fever. Further, adding amino acids to the C-terminal of α-MSH(11-13) (SEQ. ID. NO. 2) sequence can reduce or enhance antipyretic potency. It is known that Ac-[D-K11]-α-MSH 11-13-NH2 has the same general potency as the L-form of the tripeptide α-MSH (11-13) (SEQ. ID. NO. 2). However, substitution with D-proline in position 12 of the tripeptide rendered it inactive. See e.g. Holdeman, M., et. al., Antipyretic Activity of a Potent α-MSH Analog, Peptides 6, 273-5 (1985). Deeter, L. B., et. al., Antipyretic Properties of Centrally Administered α-MSH Fragments in the Rabbit, Peptides 9, 1285-8 (1989). Hiltz, M. E., Anti-inflammatory Activity of α-MSH (11-13) Analogs: Influences of Alterations in Stereochemistry, Peptides 12, 767-71 (1991).

Biological functional equivalents can also be obtained by substitution of amino acids having similar hydropathic values. Thus, for example, isoleucine and leucine, which have a hydropathic index +4.5 and +3.8, respectively, can be substituted for valine, which has a hydropathic index of +4.2, and still obtain a protein having like biological activity. Alternatively, at the other end of the scale, lysine (−3.9) can be substituted for arginine (−4.5), and so on. In general, it is believed that amino acids can be successfully substituted where such amino acid has a hydropathic score of within about +/−1 hydropathic index unit of the replaced amino acid. See, U.S. Pat. No. 5,157,023 issued to James M. Lipton issued on Oct. 20, 1992.

### EXAMPLE 2

### Use of A-MSH in Selected Sections of Intestinal Mucosa

Three sets of experiments were performed using duodenal biopsy pairs from 53 adult celiac patients and 14 normal subjects to determine: 1. a) mucosal immunoreactivity for (α-MSH and melanocortin receptors (MCRs); and b) gene expression of α-MSH precursor pro-opiomelanocortin (POMC) and MCRs; 2. a) α-MSH and cytokine production by duodenal specimens in vitro, and b) influence of synthetic α-MSH on cytokine production; and 3. a) influence of stimulation with gliadin, the subfraction of gluten that is toxic to patients with celiac disease, on α-MSH and cytokine production in vitro, and b) effect of α-MSH on gliadin-stimulated cytokine production.

Duodenal mucosa showed immunostaining for α-MSH, MC1R and MC5R. α-MSH and MC1R immunoreactivity were more intense in specimens from celiac patients. Specific mRNAs were likewise expressed. In experiments in vitro, synthetic α-MSH significantly reduced interleukin 6 (IL-6) release in supernatants of gliadin-stimulated duodenal mucosa.

The inventors postulated that an autocrine anti-inflammatory circuit based on α-MSH and its receptor(s) may occur in the celiac mucosa. To test this hypothesis, the inventors determined generation and actions of α-MSH in specimens of duodenal mucosa obtained from celiac patients. Because any effect of the peptide would be mediated by binding to its receptor(s), we determined expression of subtypes of the known melanocortin receptors (MCR). As an appraisal of the local anti-inflammatory influences of α-MSH in celiac mucosa, we determined effects of incubation of intestinal mucosa with the synthetic peptide on spontaneous and gliadin-induced cytokine production in vitro. The research plan included three separate sets of experiments performed on duodenal biopsy pairs from the same individual—celiac or normal. The first set of experiments was done to determine: a) mucosal immunoreactivity for (α-MSH and its receptors and b) gene expression of α-MSH precursor pro-opiomelanocortin (POMC) and MCRs. The second series of biopsy pairs were used to determine: a) production of α-MSH peptide and cytokines by duodenal mucosa specimens incubated for 24 h in vitro and b) influence of synthetic α-MSH on cytokine production. Finally, specimen pairs were used to investigate: a) influence of gliadin-stimulation on A-MSH and cytokine production in vitro, and b) effect of α-MSH on gliadin-stimulated cytokine production by duodenal mucosa.

Thirty-four untreated patients with celiac disease (24-55 yr, 20 F) and 19 celiac patients on a gluten-free diet (25-52 yr, 11 F) undergoing upper duodenal endoscopy were included in this research. Diagnosis was made on the basis of positive serologic tests (IgA-antiendomysial antibodies, IgG- and IgA-antigliadin antibodies), small bowel histology, clinical and histologic response to a gluten-free diet, and relapse following gluten challenge. Specimens from 14 subjects (26-66 yr, 6 F) investigated for gastrointestinal symptoms in whom endoscopic and histological findings were normal were used as controls.

Four biopsy specimens from the distal duodenum were obtained during upper gastrointestinal endoscopy. Two of such specimens were fixed in 10% formalin and paraffin embedded for standard histological examination. The remaining two specimens from each subject were available for this research and were allocated for either 1) RT-PCR and immunohistochemical studies (N=20: 9 celiac, 7 celiac on diet, 4 normal); 2) cytokine production in vitro in the presence or absence of α-MSH(N=22: 11 celiac, 6 celiac on diet, 5 normal); or 3) cytokine production in vitro after gliadin stimulation in the presence or absence of α-MSH(N=25: 14 celiac, 6 celiac on diet, 5 normal).

Blood samples (7 ml in ethylenediamine tetra-acetic acid) were drawn from an antecubital vein from all subjects for determination of circulating A-MSH. The samples were immediately centrifuged and aliquots of plasma were stored at −80° C.

POMC and MCR Gene Expression

Biopsy specimens of duodenal mucosa assigned to RT-PCR studies were snap frozen in liquid nitrogen and stored at −80° C. Total RNA was isolated from homogenized specimens using the acid phenol—guanidinium thiocyanate method (Chomczynski P, Sacchi N., Single-step method of RNA isolation by acid guanidinium thiocyanate-phenol-chloroform extraction, Anal Biochem 1987; 162:156-159), and stored at −80° C. To prevent misinterpretation due to genomic contamination, as MCR genes lack introns, total RNA was treated with amplification grade RNase-free DNase I (Life Technologies, Gaithersburg, Md.) at room temperature for 15 min. DNase I was then inactivated by adding 2.5 mM EDTA and heating at 65° C. for 10 min. First-strand cDNA synthesis was performed, using 1 μg of each RNA sample, 20 pmoles of oligo (dT)¹⁸ primer, and 200 U of MMLV reverse transcriptase (Clontech Laboratories, Palo Alto, Calif.) in a 20-μl reaction volume. PCR amplifications were performed on portions (4-10%) of each diluted (1:5) cDNA mixture in a 25-μl reaction volume containing 20 pmoles each of upstream and downstream primers, 1U of AmpliTaq DNA polymerase (Perkin Elmer Applied Biosystems, Foster City, Calif.), 200 μM dNTPs and 1-1.5 mM MgCl₂. PCR reaction mixture and temperature profile conditions were tested against positive controls for each specific mRNA in preliminary experiments. The POMC primer pair (upstream 5′-GAGGGCAAGCGCTCCTACTCC-3′, downstream 5′-GGGGCCCTCGTCCTTCTTCTC-3) generated a 260-base pair (bp) product, the MCIR pair (upstream 5′-GCCACCATCGCCAAGAACC-3′, downstream 5′-ATAGCCAGGAAGAAGACCA-3) a 416-bp product, the MC3R pair (upstream 5′-CGGTGGCCGACATGCTGGTAAGTG-3′, downstream 5′-TGAGGAGCATCATGGCGAAGAACA-3) a 461-bp product, the MC5R pair (upstream 5′-CATTGCTGTGGAGGTGTTTCT-3′, downstream 5′-GCCGTCATGATGTGGTGGTAG-3) a 357-bp product. All PCR products were resolved by 2% agarose gel electrophoresis. A glyceraldehyde-3-phosphate dehydrogenase (G3PDH) primer pair (upstream 5′-TGAAGGTCGGAGTCAACGGATTTGGT-3′, downstream 5′-CATGTGGGCCATGAGGTCCACCAC-3), generating a 980-bp product, was used for normalization.

Immunohistochemistry

Duodenal specimens assigned to immunohistochemical analysis were fixed in 10% buffered formalin and paraffin-embedded. Tissue sections (3 μm) were cut from paraffin-embedded blocks. Prior to use, sections were dewaxed, hydrated and endogenous peroxidase activity was blocked by incubation with 1% hydrogen peroxide in distilled water. After antigen retrieval using microwave irradiation at 780 W in pH 6.0 citrate buffer for 15 min for, the slides were allowed to cool at room temperature for 20 min and incubated at 4° C. overnight either with a rabbit polyclonal antibody against α-MSH (Eurodiagnostica, Malmö, Sweden) using a 1:600 dilution, or with goat polyclonal antibodies against MC1R (1:400), MC3R (1:200), or MC5R (1:700) (Santa Cruz Biotechnology, Santa Cruz, Calif.). Sections were subsequently rinsed in Tris-NaCl buffer pH 7.6 and incubated at room temperature for 30 min with biotin-conjugated donkey anti-rabbit or donkey anti-goat IgG secondary antibodies (Santa Cruz Biotechnology, Santa Cruz, Calif.). After rinsing in Tris-NaCl buffer, sections were incubated for 30 min with avidin-biotinylated horseradish peroxidase complex. The final reaction was run by incubating the sections in 0.002% 3′3′-diaminobenzidine (DAB) solution added with 0.01% hydrogen peroxide for 5 min. The sections were then washed in deionized H₂O and counterstained with Harry's hematoxylin.

α-MSH and Cytokine Production by Celiac Mucosa in Vitro

A series of 47 specimen pairs were used to evaluate cytokine production by celiac mucosa in vitro and the influence of α-MSH on such production. Twenty-two pairs were used to evaluate spontaneous production and 25 were stimulated with gliadin; both experiments were performed in the presence or absence of α-MSH. The duodenal specimens were immediately placed in a 6-well culture plate and incubated in 5% CO₂ atmosphere at 37° C. for 24 h with either 1) 1.5 ml medium (RPMI 1640 supplemented with 10% fetal bovine serum, 2 mM glutamine, 10 mM HEPES buffer), 50 U/ml penicillin, 50 μg/ml streptomycin or 2) medium and (α-MSH 10⁻⁵ M (kindly provided by Dr. Renato Longhi, CNR, Milano) in the same final volume. To estimate the influence of α-MSH on gliadin-stimulated cytokine production, each of the two specimens from the same subject were incubated in the experimental conditions described above with either 1) peptic/tryptic digest of commercial gliadin (Sigma-Aldrich, St. Louis, Mo.) or 2) gliadin and synthetic α-MSH 10⁻⁵ M. After 24 h incubation, the culture medium was removed, distributed in 4 aliquots and stored at −80° C. for measurement of interleukin 1β (IL-1β), IL-6, and TNF-α. One of the aliquots from biopsies not treated with α-MSH (medium alone or gliadin-stimulated) was used to measure endogenous production of α-MSH by duodenal mucosa.

Assays

α-MSH was measured using a double-antibody radioimmunoassay (RIA) method (Eurodiagnostica, Malmo, Sweden); the sensitivity calculated from a decrease in binding of 2 SD in the zero standard was 4.8 pg/ml and the cross-reactivity with other POMC peptides (ACTH [1-24], ACTH [1-39], β-MSH, γ-MSH) is <0.002%. IL-1β, IL-6, and TNF-α were determined using commercial ELISA methods (Amersham Pharmacia Biotech, Little Chalfont, UK).

Statistical Analysis

Values were expressed as means±SEM. Two-sample comparisons were performed using Student's t test. Multiple comparisons were performed using analysis of variance (ANOVA) followed by Bonferroni t test for specific comparisons (SigmaStat Statistical Software, Jandel GmbH, Erkrath, Germany). Probability values <0.05 were considered significant.

α-MSH Production by Celiac Mucosa in Vitro

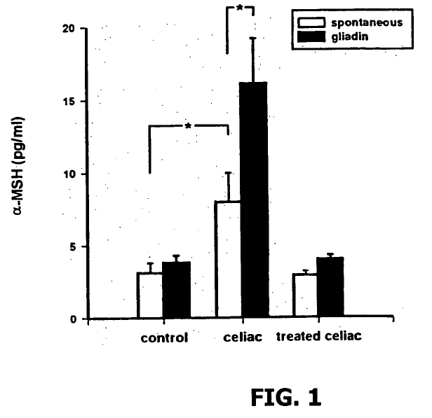

Supernatants of duodenal mucosa incubated for 24 h in vitro showed a small but clearly detectable spontaneous production of α-MSH, which was significantly greater (p<0.05) in celiac patients (n=11) relative to both normal subjects (n=5) and treated celiac patients (n=6) (FIG. 1). Conversely, such production was similar in normal controls and treated celiac patients. When mucosal specimens were incubated with gliadin for 24 h, mean α-MSH release was enhanced only in patients with untreated celiac disease (FIG. 1) (p<0.05). The low production of peptide observed in normal duodenal mucosa or in treated celiac patients was not altered by gliadin.

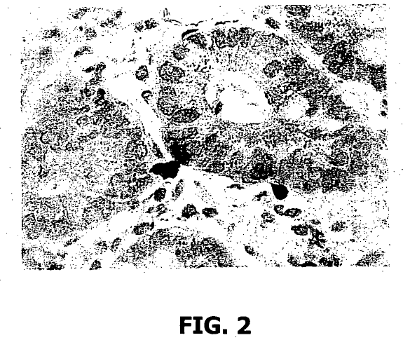

α-MSH immunoreactive cells were present in duodenal mucosa specimens of untreated celiac patients (FIG. 2). Immunoreactivity was mainly localized in endocrine-like cells, although scattered cells in the lamina propria were likewise immunoreactive. A few immunoreactive cells were occasionally found also in normal duodenal specimens and in treated celiac patients (not shown).

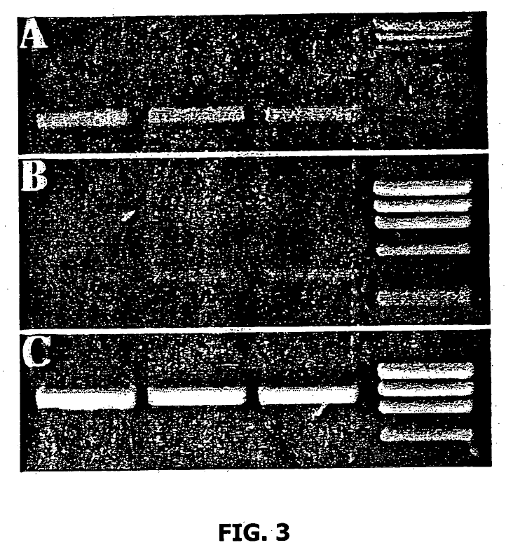

Presence of immunoreactive α-MSH was confirmed by expression of mRNA for the α-MSH precursor POMC in duodenal mucosa homogenates (FIG. 3A).

Plasma concentration of α-MSH in celiac patients was within the normal range, although untreated patients had slightly higher concentrations relative to the other two groups (20.46±3.0, 28.4±5.5, 22.35±2.6, in normal, untreated celiac and treated celiac respectively, p>0.05).

MCRs Expression in Duodenal Mucosa

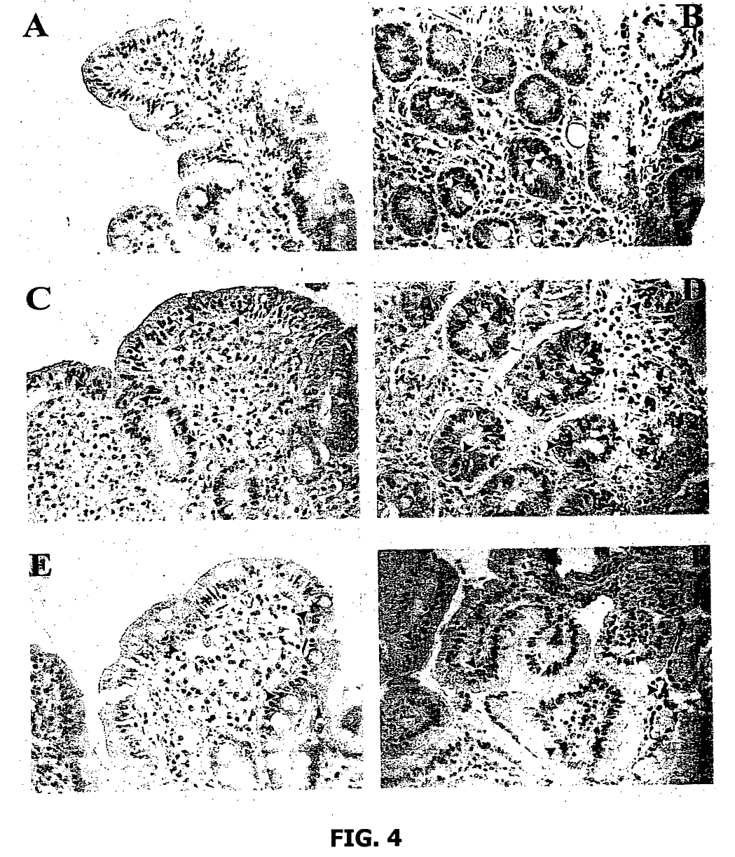

Immunohistochemical analysis of duodenal specimens for MC1R showed positive staining of goblet cells in both celiac and normal subjects. However, the number and distribution pattern of positive cells in non-celiac and celiac individuals was substantially different. In non-celiac mucosa, MC1R immunoreactive cells were fewer and localized solely to the base of glands (FIGS. 4A, 4B). Immunostaining occurred mainly in the outer margins of the mucous globule. No immunoreactivity was found on the villous surface in normal mucosa. In celiac mucosa, the majority of goblet cells were immunoreactive for MC1R; such cells were distributed along the entire length of glandular tubules and on the surface of flattened villi (FIGS. 4C, 4D). Further, there was a substantial difference in intensity of immunostaining; it was stronger in cells of celiac mucosa and much weaker in goblet cells of non-celiac mucosa. MC1R immunoreactivity in celiac patients treated with a gluten-free diet showed the same pattern of distribution observed in patients with active disease, involving the villous surface but with less intensity of immunostaining (FIGS. 4E, 4F).

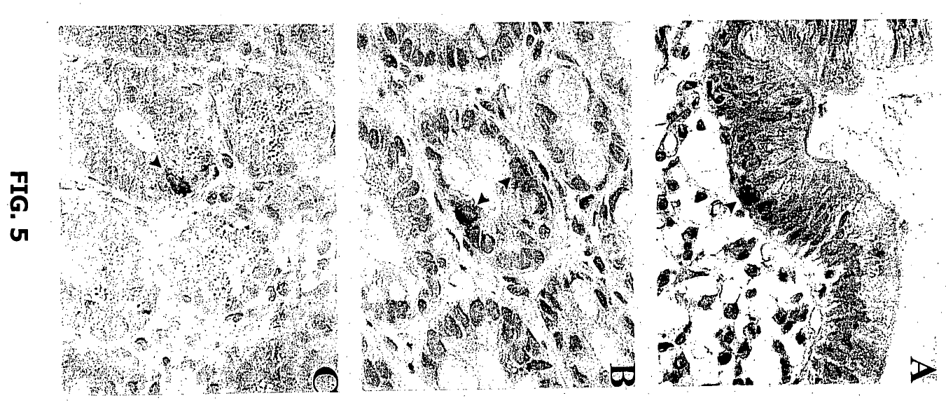

Scattered cells showed staining with the antiserum against MC5R. Such cells were distributed in both glandular crypts and mucosal villi; the pattern of immunostaining was similar to that usually shown by neuroendocrine cells, being more evident in the subnuclear area of the cytoplasm (FIG. 5). There was no substantial difference between specimens from celiac and control subjects.

Presence of MC1R in duodenal mucosa was confirmed by expression of mRNA for this receptor subtype (FIG. 3B). No mRNA for MC5R was found. This is not surprising since there were very few immunoreactive cells. No expression of the receptor subtype MC3R was found using either immunohistochemical or RT-PCR techniques.

Influence of α-MSH on Cytokine Production in Vitro

Non-stimulated 24 h production of IL-1β, IL-6, and TNF-α by celiac mucosa in vitro was low or undetectable and there was no change after addition of α-MSH to the incubation medium.

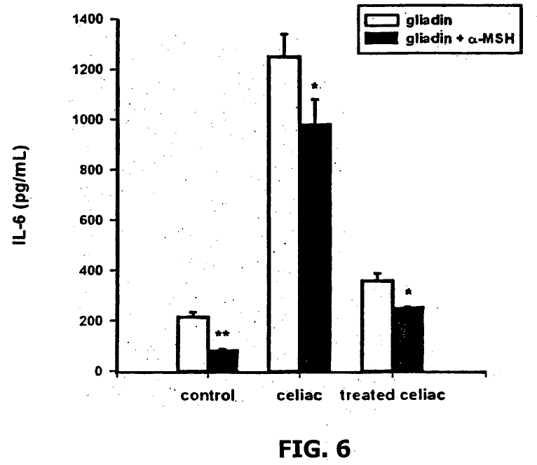

Twenty-four hour incubation of duodenal specimens in vitro with gliadin significantly enhanced IL-6 release in the supernatants (FIG. 6). Concentration of the cytokine in presence of gliadin was significantly greater in celiac patients relative to patients with inactive disease or controls (n=5 controls, 14 untreated celiac, and 6 treated celiac patients). When gliadin-stimulated duodenal specimens were co-incubated with α-MSH, there was a statistically significant inhibition of IL-6 release in all groups. IL-1β and TNF-α were low or undetectable even after gliadin stimulation in all groups (not shown).

From the foregoing, it will be appreciated that specific embodiments of the invention have been described herein for purposes of illustration, but that various modifications may be made without deviating from the spirit and scope of the invention. Accordingly, the invention is not limited except as by the appended claims. The preceding Examples are intended only as examples and are not intended to limit the invention. It is understood that modifying the examples above does not depart from the spirit of the invention. It is further understood that the each example may be applied on its own or in combination with other examples.

1. A method of treatment of malabsorption conditions of the gastrointestinal tract comprising: diagnosis of a malabsorbtive condition of the gastrointestinal tract in a patient; and administering to the patient a therapeutically effective amount of α-MSH in combination with a therapeutically effective amount of artichoke leaf extract.

2. The method of claim 1 wherein the malabsorptive conditions of the gastrointestinal tract are selected from the group consisting of celiac disease, tropical sprue, Whipple's disease, bacterial overgrowth, Crohn's disease, ulcerative colitis, allergic gastroenteritis, lymphoma-associated diffuse enteritis and acute infectious enteritis.

3. The method of claim 1 wherein the therapeutically effective amount of α-MSH and/or its derivatives is at least about 25 mg/kg of body weight of the patient.

4. The method of claim 1 wherein the therapeutically effective amount of artichoke leaf extract is at least about 300 to 1000 mgs.

5. The method of claim 1 wherein the administration is selected from the group consisting of oral, mucous membrane, anal and parenteral.

6. A method of treating celiac disease comprising: diagnosing a patient with celiac disease; administering to the patient a therapeutically effective amount of α-MSH in combination with a therapeutic amount of artichoke leaf extract.

7. The method of claim 6 wherein the therapeutically effective amount of α-MSH and/or its derivatives is at least about 25 mg/kg of body weight of the patient.

8. The method of claim 6 wherein the therapeutically effective amount of artichoke leaf extract is at least about 300 to 1000 mgs.

9. The method of claim 6 wherein the administration is selected from the group consisting of oral, mucous membrane, anal and parenteral.

10. A method of prevention of complications associated with malabsorption conditions of the gastrointestinal tract consisting of: diagnosis of complications associated with malabsorption conditions of the gastrointestinal tract in a patient; and administering to the patient a therapeutically effective amount of α-MSH in combination with artichoke leaf extract.

11. The method of prevention of claim 10 wherein the malabsorption conditions of the gastrointestinal tract are selected from the group consisting of celiac disease, tropical sprue, Whipple's disease, bacterial overgrowth, Crohn's disease, ulcerative colitis, allergic gastroenteritis, lymphoma-associated diffuse enteritis and acute infectious enteritis.

12. The method of prevention of claim 10 wherein the therapeutically effective amount of α-MSH and/or its derivatives is at least about 25 mg/kg of body weight of the patient.

13. The method of prevention of claim 10 wherein the artichoke leaf extract is at least about 300 to 1000 mgs.

14. The method of prevention of claim 10 wherein the administration is selected from the group consisting of oral, mucous membrane, anal and parenteral.

15. A method of preventing complications related to celiac disease comprising: diagnosing a patient with celiac disease; administering to the patient a therapeutically effective amount of α-MSH in combination with a therapeutic amount of artichoke leaf extract.

16. The method of prevention of claim 15 wherein the therapeutically effective amount of α-MSH and/or its derivatives is at least about 25 mg/kg of body weight of the patient.

17. The method of prevention of claim 15 wherein the artichoke leaf extract is at least about 300 to 1000 mgs.

18. The method of prevention of claim 15 wherein the administration is selected from the group consisting of oral, mucous membrane, anal and parenteral.

19. A pharmaceutical composition for the treatment of malabsorbtion conditions omprising α-MSH in combination with derivatives of α-MSH and artichoke leaf extract.

20. The composition of claim 19 wherein the α-MSH is at least about 25 mg/kg of a recipient's body weight.

21. The composition of claim 20 wherein the α-MSH is a combination of α-MSH and derivatives of A-MSH.

22. The composition of claim 21 wherein the derivative of α-MSH is VPKccKPV (SEQ ID NO: 5).

23. The composition of claim 20 wherein the artichoke leaf extract is at least about 300 to 1000 mgs.
